(19) United States
(12) Patent Application Publication
(10) Pub. No.: US 2026/0321351 A1
(43) Pub. Date: Oct. 15, 2026

(54) METHODS AND SYSTEMS FOR CANCER-ENRICHED MOTIF DISCOVERY FROM SPLICING VARIATIONS IN TUMOURS

(71) Applicant: NEC Laboratories Europe GmbH, Heidelberg (DE)

(72) Inventors: Israa ALQASSEM, Heidelberg (DE); Filippo GRAZIOLI, Heidelberg (DE); Anja MOESCH, Heidelberg (DE)

(21) Appl. No.: 19/101,255

(22) PCT Filed: Aug. 12, 2022
(86) PCT No.: PCT/EP2022/072739

§ 371 Date: Feb. 5, 2025

(51) Int. Cl. G16B 40/20; G16B 20/20

(52) U.S. Cl. CPC G16B 40/20; G16B 20/20

(57) ABSTRACT

A computer-implemented method of training a machine learning model for identifying sequence motifs indicative of a disease from alternative splicing events. A list of nucleotide sequences is obtained, where each sequence of the list of nucleotide sequences represents a sequence of an alternative splicing event. A label is obtained for each sequence, where the label indicates whether the each sequence is associated with a healthy sample or a diseased sample. A machine learning model is trained on the list of nucleotide sequences and respective labels to classify alternative splicing events from corresponding sequences based on whether they occur in healthy or diseased samples.

### CROSS REFERENCE TO RELATED APPLICATIONS

This application is a U.S. National Phase application under 35 U.S.C. § 371 of International Application No. PCT/EP2022/072739, filed on Aug. 12, 2022. The International Application was published in English on Feb. 15, 2024 as WO 2024/032909 A1 under PCT Article 21(2).

### FIELD

Tumours can alter the human transcriptome and produce neoantigens (i.e., foreign proteins) which are absent from normal tissues. Cancer neoantigens can be detected by the immune system, hence they have been leveraged in immunotherapies to enhance T cell reactivity against tumour cells, i.e., when T cells get activated by a neoantigen, they can eliminate the affected tumour cells. Previous approaches to identify candidate neoantigens focus on neoantigens derived from somatic mutations, however recent research shows that cancer specific alternative splicing (AS) events represent an additional source of neoantigen candidates. Through AS, a single gene can produce multiple transcripts that combine exons in alternative ways. These transcripts can potentially translate into different proteins. Similarly, cancer specific AS events may alter gene transcripts, enrich certain (DNA) sequence motifs, and generate neoantigens.

### BACKGROUND

State of the art research has examined cancer motifs for predicting transcription factor binding sites. Other approaches have examined cancer motifs from the perspective of mutational patterns that are prevalent in cancer genome. However, they have mainly addressed cancer mutation signatures, i.e., nucleotide sequence contexts upstream and/or downstream of certain cancer mutations.

Over recent years various research has focused on neoantigens generated from somatic DNA alterations such as nonsynonymous point mutations, insertion-deletions, and frameshift mutations, however less attention has been paid to neoantigens that are derived from splicing variations in tumours.

A few studies have addressed the problem of identifying AS-based neoantigens, for example, Kahles et al. “Comprehensive analysis of alternative splicing across tumors from 8,705 patients” Cancer cell 34.2 (2018): 211-224, conducted a pan-cancer analysis of AS events across 32 different cancer types from 8,705 patients. In this study they leveraged RNA and whole-exome sequencing data of tumour and healthy samples from The Cancer Genome Atlas (TCGA) and The Genotype-Tissue Expression (GTEx) portal. In their workflow, they identified significant changes in AS in tumour compared to normal tissues, and they enumerated novel splicing junctions in TCGA samples (i.e., neojunction) which did not naturally occur in GTEx normal samples. Additionally, they examined whether the discovered neojunction are translated into proteins. They were able to experimentally verify a subset of neojunction-derived peptides as potential cancer neoantigens.

ASNEO is another recent computational pipeline for identifying personalised AS-based neoantigens from RNA-seq data and is described in Zhang, Zhanbing, et al. “ASNEO: identification of personalized alternative splicing based neoantigens with RNA-seq.” Aging (Albany NY) 12.14 (2020): 14633. ASNEO identifies novel gene isoforms based on novel splicing junctions that are only present in tumours. Then, it translates the identified novel isoforms into novel proteins. From the set of novel proteins, ASNEO generates a set of peptides as a potential source of cancer neoantigens. The ASNEO pipeline was verified on two published immunotherapy-treated cohorts, and the two key findings were (i) AS-based neoantigens have higher immune score compared to neoantigens which are identified from somatic DNA mutations, and (ii) AS-based neoantigens have a potential for predicting patient survival patterns.

The above two methods rely merely on identifying novel splicing junctions in cancer samples as potential source for neojunctions. Identifying novel splicing junctions in tumour samples and verifying that such junctions are not present in normal tissues can be time-consuming steps. Further, identifying and verifying novel cancer junctions using traditional approaches can be difficult to generalise when new patients' data becomes available.

In Smart, Alicia C., et al. “Intron retention is a source of neoepitopes in cancer.” Nature biotechnology 36.11 (2018): 1056-1058, there was proposed an in-silico approach to identify neoepitopes derived from tumour intron retention events. In-vivo verification was performed where they show, using mass spectrometry, that the neoepitopes derived from intron retention events are presented on MHC I of the cancer cells.

It thus remains a significant industry goal to provide a comprehensive approach for eliciting cancer-enriched motifs.

### SUMMARY

In an embodiment, the present disclosure provides a computer-implemented method of training a machine learning model for use in identifying sequence motifs indicative of a disease from alternative splicing events. The method includes obtaining a list of nucleotide sequences, each sequence of the list of nucleotide sequences representing a sequence of an alternative splicing event, obtaining a label for each sequence, the label indicating whether the each sequence is associated with a healthy sample or a diseased sample, and training a machine learning model on the list of nucleotide sequences and respective labels to classify alternative splicing events from corresponding sequences based on whether they occur in healthy or diseased samples.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 shows a schematic of a high-level methodology, according to examples of the disclosure;

FIG. 2 shows a schematic workflow of the proposed approach, according to examples of the disclosure;

FIG. 3 shows a schematic convolutional neural network according to examples of the disclosure;

FIG. 4 shows example benchmarking plots, according to examples of the disclosure;

FIG. 5 shows a method according to an embodiment of the present disclosure; and

FIG. 6 shows a schematic workflow of obtaining candidate neoantigens or neoepitopes from cancer-enriched motifs, according to examples of the disclosure.

### DETAILED DESCRIPTION

According to an aspect of the invention there is provided a computer-implemented method of training a machine learning model for use in identifying sequence motifs indicative of a disease from alternative splicing events, the method comprising: obtaining a list of nucleotide sequences, each sequence representing; obtaining a label for each sequence, the label indicating if the sequence is associated with a healthy sample or a diseased sample; and, training a machine learning model on the list and respective labels to classify alternative splicing events from their sequences based on whether they occur in healthy or diseased samples.

Preferably each sequence represents the DNA sequence of exons in mature messenger RNA (mRNA) or the constitutive and alternative exons of an alternative splicing event, such sequences are exonic sequences. Preferably the nucleotide sequences are DNA sequences such that the sequence motif is a DNA sequence motif and training a machine learning model on the list and respective labels classifies alternative splicing events from their DNA sequences based on whether they occur in healthy or diseased samples. Preferably, an AS event is considered cancerous if it uniquely occurs in one or more cancer sample and it does not occur in any other healthy sample. To obtain a list of cancer-specific AS events, all alternative splicing events in tumour samples may be analysed, followed by filtering out of the subset of events that are common in healthy and cancer tissues.

Preferably each label indicates if the sequence is associated with a sample of healthy tissue or cancerous tissue and the machine learning model is trained to classify alternative splicing events from their sequences based on whether they occur in healthy or cancer tissues.

The method may further comprise: identifying, by interpreting the trained model, features of the sequences contributing to indicate whether the sequences occur in healthy or diseased samples; selecting one or more high attribution regions of one or more of the sequences based on the identified features, wherein the high attribution region indicates a region of the sequence having positively contributing features; and, outputting a sequence motif based on the one or more high attribution regions. The DNA sequence motif may be thought of as a high attribution sub-sequence of a nucleotide sequence which contributes to whether or not a DNA sequence is associated with an alternative splicing event occurring in a diseased sample.

According to the concepts described herein it is possible to identify unique cancer-enriched motifs from tumour-specific splicing variations, where those motifs can provide a valuable source for cancer diagnosis and act as a potential source of neoantigens.

Methods and systems described herein may be utilised to identify cancer-enriched motifs from splicing variations in tumours and are not limited to so-called mutation signatures. Mutations in tumour can disrupt alternative splicing and result in novel splicing variations. Thus, it is proposed that considering DNA mutations which are present in tumour samples enables implementations to gain a comprehensive understanding of splicing variations and the accompanied cancer-enriched motifs. Thus, proposed systems and methods provide a more comprehensive view for eliciting cancer-enriched motifs than state of the art techniques. Motifs based on tumour splicing variations have not been hitherto examined.

Examples set out herein can identify significantly enriched motifs in different types of alternative splicing events. The alternative splicing events may be selected from a group comprising: exon skipping; alternative donor sites; alternative acceptor sites; intron retention; mutually exclusive exons, and other more complex splicing patterns. Proposals set out herein are not limited to specific alternative splicing events, such as intron retention events, and the computational pipeline can identify significantly enriched motifs in all types of alternative splicing events.

Preferably each nucleotide sequence represents exonic sequences of alternative splicing events.

By obtaining we mean that the sequences and labels may be retrieved from a data store or otherwise generated and assigned by the method, such as in a pre-processing step. For example, the method may comprise obtaining the list of nucleotide sequences from a store and assigning a label to each sequence. The label may be a ground truth label. The method may comprise retrieving a list of nucleotide sequences and enumerating AS events based on sequencing read evidence.

The regions may each have a length such that the regions are contiguous sub-sequences of nucleotides present in the DNA sequence. Preferably the length of the region may be above a minimum threshold, preferably the length may be greater than or equal to 5.

In certain implementations each feature may be a nucleotide of the sequence. In this way the trained model learns which nucleotides positively contribute to the occurrence of the alternative splicing event in the diseased sample. Alternatively, the features may be groups of nucleotides or based on an analysis of the nucleotide sequence.

The method may comprise interpreting predictions of the trained model using a saliency approach. Accordingly, the selecting may comprise: generating a feature attribution score for each nucleotide in a respective sequence based on a contribution of that nucleotide to the classification; and, selecting a region of the sequence based on the score for each nucleotide. The feature attribution score may be assigned based on Integrated Gradients, i.e. applying an Integrated Gradients interpretation approach to the trained model. Alternatively, the trained model may be interpreted using Guided Backpropagation and Occlusion Maps. Other suitable interpretation methods may be used to assign a score for each feature for subsequent identification of motifs based on the features and the relationships between the inputs and the outputs of the model.

The selecting a region of the sequence based on the score for each nucleotide may comprise: comparing the score for each nucleotide in a candidate region to a threshold; calculating an average of scores for the nucleotides in a candidate region; calculating an average of scores for the sequence and comparing the score for each nucleotide in a candidate region to the average of scores for the sequence.

According to an aspect of the invention there may be provided a method of identifying sequence motifs indicative of a disease from alternative splicing events, the method comprising: retrieving features of sequences contributing positively to indicate whether a nucleotide sequence representing a sequence of an alternative splicing event occurs in healthy or diseased samples; retrieving an attribution score for each feature; comparing a nucleotide sequence with the features; and identifying a motif in the sequence based on the score for each feature in the nucleotide sequence.

According to an aspect of the invention there may be provided a method of identifying sequence motifs indicative of a disease from alternative splicing events, the method comprising: retrieving an attribution score of each nucleotide in an alternative splicing sequence to indicate whether a nucleotide and its neighbouring region represent a subsequence that is more likely to occur in healthy or diseased samples; and identifying a motif in the sequence based on the scores of contiguous regions in the nucleotide sequence that meet various predefined thresholds. Such regions may be referred to as disease-enriched motifs.

The selecting may comprise applying a hypergeometric test to the one or more high attribution regions to obtain significant regions present in the sequences associated with a diseased sample label and filter out regions common to the nucleotide sequences associated with a diseased sample label and the nucleotide sequences associated with a healthy sample label.

The method may further comprise merging a plurality of the selected one or more regions using pairwise sequence alignment. Merging the regions reduces the total number of motifs, provides a list of representative motifs and improves the statistical likelihood of a motif being relevant. The merging step may reduce redundancy. For example, two motifs can have the same sequence in the middle but one is shifted to the right or left, another example when one motif is a subset of another motif so we align and merge them to obtain a list of representative motifs.

The method may further comprise filtering the selected one or more regions based on a number of occurrences of each region in the sequences associated with a diseased sample label. Filtering in this way improves the statistical likelihood of the region being relevant and helps in eliminating artifact and uncommon motifs.

In preferred implementations, the DNA sequence motif may be output together with a corresponding genomic coordination and a list of genes and transcripts where the motif occurs.

The DNA sequence motif can be leveraged for cancer diagnosis and act as a potential source for cancer neoantigens, i.e., cancer therapy or immunotherapy.

The machine learning model may be any suitable machine learning or statistical model. In examples the machine learning model may be a derivable parametric model. The machine learning model is a neural network, preferably a multi-kernel-size convolutional neural network for 1-dimensional signals. The machine learning model may be referred to as a deep learning model.

Thus, in preferred implementations methods disclosed herein use a deep learning-based approach for classifying alternative splicing events in cancerous and normal tissues as opposed to existing approaches which rely on enumerating all novel splicing in tumours by one-to-one comparisons to all splicing events that occur in normal samples. Using a deep learning-based model helps improve precision in identifying common cancer-specific splicing patterns in pan-cancer analysis.

Benchmarking analysis has shown the proposed multi-kernel-size convolutional neural network for 1-dimensional signals has a shorter training time and provides good performance in terms of precision, F1 and Matthews correlation coefficient (MCC) scores when compared to state of the art models.

The method may further comprise applying the trained model to one or more unseen nucleotide sequences to identify if the nucleotide sequence corresponds to a healthy or disease specific alternative splicing event. For example, a normal or cancer specific alternative splicing event. In this way the approach set out herein generalises to new unseen alternative splicing events and does not require repetitive manual comparisons between tumour and healthy samples. The model may be trained once on large number of samples such that it is able to distinguish normal and cancer specific AS events with an F1 and recall scores greater than 90%.

Preferably the method may further comprise identifying a neoantigen candidate or neoepitope candidate for immunotherapy based on the sequence motif.

Alternative methods rely on identifying cancer neoantigens based either on cancer-specific splicing junctions or somatic DNA alterations. Whereas the present system identifies neoantigens based on cancer-enriched motifs from novel splicing variations in tumours. Alternative methods do not scale easily with additional datasets and require more computational resources to aid in the comparisons between alternative splicing events in normal and tumour samples, whereas the present system includes a trained model that can efficiently classify cancerous and healthy AS events from their DNA sequences.

Additionally, according to an aspect of the invention there may be provided a method of identifying a neoantigen candidate or neoepitope candidate for immunotherapy based on a sequence motif output according to any of the above aspects or implementations.

Obtaining a list of alternative splicing-derived neoepitope or neoantigen candidates may involve the steps of: extracting k-mer peptides (where k≥9) covering each of the cancer-enriched motif, filtering out the peptides that are present in a non-cancer proteomic dataset; confirming potential neoepitopes or neoantigens in protein mass spectrometry (MS) databases obtained from various tumour types (such as Clinical Proteomic Tumor Analysis Consortium (CPTAC) MS data); and running an existing tool to predict whether the neoepitopes or neoantigens bind to major histocompatibility complex (MHC) to check their immunogenicity. Neoepitopes or neoantigens which provoke an immune response can be leveraged in cancer immunotherapies. These verification steps are described in literature (for example, Kahles et al. “Comprehensive analysis of alternative splicing across tumors from 8,705 patients” Cancer cell 34.2 (2018): 211-224). Furthermore, peptides derived from newly identified alternative splicing events can be leveraged in identifying novel neoepitope or neoantigen candidates in MS data from eluted peptide-MHC complexes that have not been previously detected and are thus not present in standard MS reference databases. Finally, the candidate neoepitopes or neoantigens can be leveraged in cancer immunotherapy vaccinations to help immune system recognise such epitopes or antigens and eliminate cancer cells which produce them.

According to an aspect of the invention there may be provided a method of creating a vaccine, comprising: selecting one or more predicted immunogenic candidate amino acid sequences covering a sequence motif output according to any of the above aspects or implementations for inclusion in a vaccine; and synthesising the one or more amino acid sequences or encoding the one or more amino acid sequences into a corresponding DNA or RNA sequence and/or incorporating the DNA or RNA sequence into a genome of a bacterial or viral delivery system to create a vaccine.

Further, according to an aspect of the invention there may be provided a method of predicting likelihood of a tissue sample being cancerous, comprising: retrieving a sequence of a tissue sample; analysing the sequence of the tissue sample for similarity to a sequence motif output according to any of the above aspects or implementations; and, predicting likelihood of a tissue sample being cancerous based on the analysis.

According to further implementations of the invention, each label may indicate if the sequence is positively associated with an inherited disease and the machine learning model may be trained to classify alternative splicing events from their sequences based whether they relate to an inherited disease. The inherited disease may for example be Autism Spectrum Disorder or Mendelian disorders. Identifying significant motifs from alternative splicing events in inherited diseases enables the development of potential treatments for such disorders and a better understanding of their origins causes and diagnosis. Concepts set out herein can be used to identify significant motifs from novel splicing variations in inherited diseases.

According to an aspect of the invention there is provided a computer-implemented method for identifying DNA sequence motifs indicative of a disease from alternative splicing events, the method comprising: obtaining a list of nucleotide DNA sequences, each sequence representing a sequence of an alternative splicing event; obtaining a label for each sequence, the label indicating if the sequence is positively associated with a healthy sample or a diseased sample; training a machine learning model on the list and respective labels to classify alternative splicing events from their DNA sequences based on whether they occur in healthy or diseased samples; identifying, by interpreting the trained model, features of the sequences contributing positively to indicate whether the sequences occur in healthy or diseased samples; selecting one or more high attribution regions of one or more of the sequences based on the identified features, wherein the high attribution region indicates a region of the sequence having positively contributing features; generating a DNA sequence motif based on the one or more high attribution regions; and, identifying a neoantigen candidate or neoepitope candidate for immunotherapy based on the DNA sequence motif, or comparing the motifs to a DNA sequence to predict presence of cancer in a tissue based on alternative splicing events.

According to an aspect of the invention there may be provided a method to identify unique cancer-enriched motif from alternative splicing events, the method includes following steps: a) collect DNA sequences of AS events obtained from healthy and tumour samples and assign labels to each sequence, i.e., cancerous, or healthy; b) using a deep learning-based approach to train a derivable parametric model to classify AS events based on whether sequence occur in normal or cancer tissues; c) to interpret the predictions of the selected model, using a saliency approach to assign attribution value i.e. positive value to the nucleotide that contribute positively to the cancerous class and vice versa; d) selecting high-attribution regions where all nucleotide within the region has attribution scores greater than a threshold value; e) applying a hypergeometric test to only keep significantly enriched motifs in cancer specific AS sequences; f) merging similar motifs and filtering less frequent motifs; and, g) the output is a list of cancer-enriched motifs which can be used for cancer diagnostics to identify cancer neoantigens.

According to an aspect of the invention a computer readable medium may be provided having computer executable instructions stored thereon for implementing the method of any of the above aspects of the invention.

According to an aspect of the invention a system may be provided, the system comprising at least one processor in communication with at least one memory device, the at least one memory device having stored thereon instructions for causing the at least one processor to perform a method according to any of the above aspects.

Over the past decade, the availability of sequencing technologies has facilitated the analysis of tumour genomes and paved the way towards deeper understanding of tumour evolution. However, identifying neoantigens for targeted cancer immunotherapies is still an ongoing challenge in cancer genomics.

A stated aim of methods and systems provided herein is to identify unique cancer-enriched motifs from tumour-specific splicing variations, where those motifs can provide a valuable source for cancer diagnosis and act as a potential source of neoantigens. A method and system is presented for identifying a set of DNA regions of over-represented motifs from dysregulation of alternative splicing in tumour transcriptomes.

Alternative splicing (AS) plays an important role in cancer development and progression. Recent research showed that tumours have approximately 30% more alternative splicing events compared to normal tissues. Therefore, splicing variations and the resulting over-represented motifs provide a valuable source for cancer diagnosis and treatment. Neoantigens derived from tumour splicing variations can be leveraged in the development of novel immunotherapies such as personalised cancer vaccines.

Throughout the present document the terms neoantigens neoepitopes may be used interchangeable to refer to altered amino acid sequences of proteins in tumour cells. Neoantigens or neoepitopes can be recognised by the immune system and trigger an immune response to cancer.

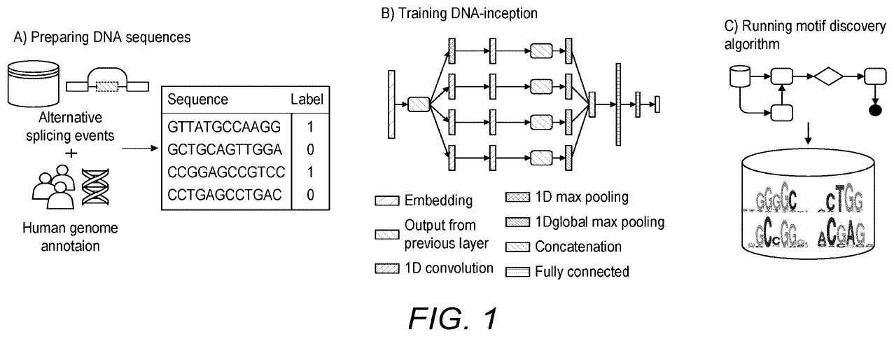

FIG. 1 illustrates a high-level schematic illustration of the steps of the proposed pipeline. In an initial step, the DNA sequences are prepared. Next, a machine learning model is trained on the prepared sequences before a motif discovery algorithm is employed which aims to interpret the trained model to identify regions of the input DNA sequences which contribute to the classification of cancerous of healthy. These regions are then considered to be the cancer enriched motifs as they occur in DNA sequences relating to alternative splicing events associated with cancerous tissue.

Initially the steps of data collection and model training are performed. At step 101, sequencing data of healthy and tumour samples is collected. Based on sequencing read evidence, the workflow prepares a list of DNA sequences of various AS events (e.g., exon skipping, alternative acceptor site, alternative donor site, intron retention, etc.) and assigns a ground truth label to each sequence, i.e., cancerous, or healthy. A suitable table showing a list of sequences and their respective labels is illustrated in FIG. 1.

Next a derivable parametric model is trained to classify AS events based on whether they occur in normal or cancer tissues. FIG. 1 illustrates the model as a convolutional neural network having a certain configuration. Details of the configuration of the model and how it is trained will be provided below. It will be understood however that what is important is that the model is trained to classify RNA splicing in cancerous and normal tissues as opposed to existing approaches which rely on enumerating all novel splicing in tumours by one-to-one comparisons to all splicing events that occur in normal samples. The model should be suitable to learn how the input DNA sequences relate to the respective labels and be capable of later interpretation.

As indicated, in a final step, the motifs are to be discovered from the trained model by interpreting the predictions of the selected model. The motif discovery algorithm uses a saliency approach to an assign attribution value to each input nucleotide in cancer specific AS events based on the model predictions.

In examples, for each cancerous AS event, the workflow finds DNA regions of length≥M where each nucleotide in the region has an attribution value higher than a predefined threshold. We refer to those DNA regions here as sequence motifs.

The workflow then: applies a hypergeometric test to only keep significantly enriched motifs in cancer specific AS sequences; uses an efficient algorithm for sequence pairwise alignment to merge similar motifs; and filters less frequent motifs.

The final motif list may be referred to as cancer-enriched motifs. This list can be leveraged further downstream analyses to identify cancer neoantigens, as explained below.

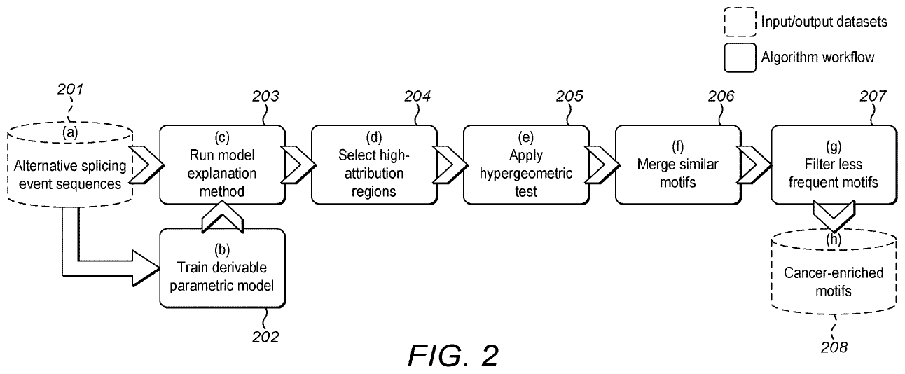

FIG. 2 shows a schematic workflow of the pipeline of the proposed system in more detail, breaking down the three steps of FIG. 1 into more detailed technical blocks.

At step 201, a set of alternative splicing event sequences are collected. The input to the system comprises DNA sequences of alternative events that are obtained from healthy and tumour samples. Various AS events are enumerated, for example exon skipping, alternative acceptor site, alternative donor site, intron retention, etc., based on sequencing read evidence. A list of nucleotide sequences X is created to represent exonic sequences of AS events. For each sequence x∈X, a label or a value is assigned y∈Y. For a binary classification problem, labels are assigned based on whether an event occurs in healthy or tumour sample, y∈{healthy, cancerous}. For a regression problem, the system is capable of supporting a continuous labelling scheme y∈[0,1], e.g., to predict exon-inclusion ratios (or equivalently percent spliced in) in healthy and cancerous AS events. Exon-inclusion ratios can be quantified from sequencing reads.

Although alternative splicing occurs at the RNA level, preferably the input to the system is the corresponding DNA sequences obtained by replacing Uracil (U) nucleotides in the RNA with Thymine (T) nucleotides.

The input sequences may be retrieved from a data store already pre-processed, or may be processed by the workflow. For example, the sequence may be retrieved from a store and the label assigned based on sequence read data or alternatively the sequences may be stored already associated with a suitable label. Examples of suitable data sets that may be utilised with the present concepts include the ExonSkipDB which is a resource for cancer and drug research communities to identify therapeutically targetable exon skipping events and contains exon skipping events from 14,272 genes based on RNA-seq and whole-exome sequencing evidence. Additional resources may be used such as The Cancer Genome Atlas (TCGA) and Genotype-Tissue Expression (GTEx).

At step 202, a derivable parametric model is trained. In this step the workflow trains a derivable parametric model using the data provided in the previous step, i.e. the input sequences and labels illustrated in FIG. 1. The derivable parametric model models the probability P_{θ}(y|x), where θ represents the model parameters. The model can be, but is not limited to, a neural network. The training is performed using for example stochastic gradient descent to minimise a predefined error metric between predicted and ground truth values. The training stops when a user-defined criteria are achieved, e.g., validation recall exceeds certain threshold after predefined number of iterations (or epochs).

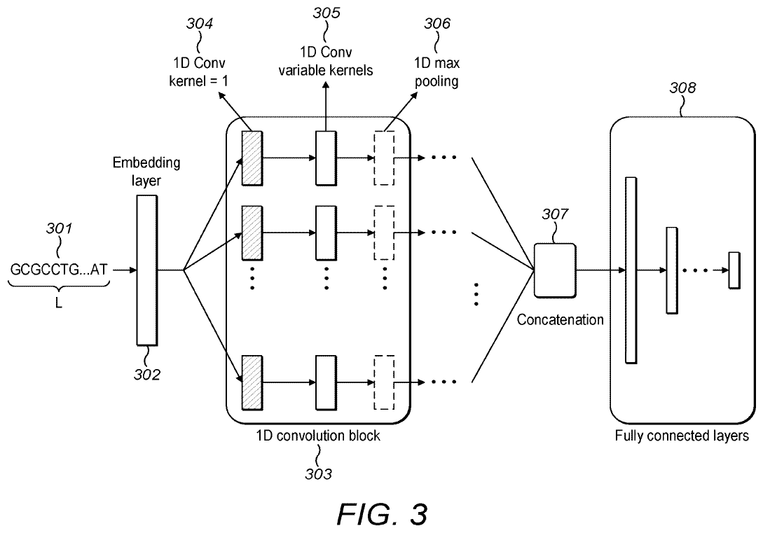

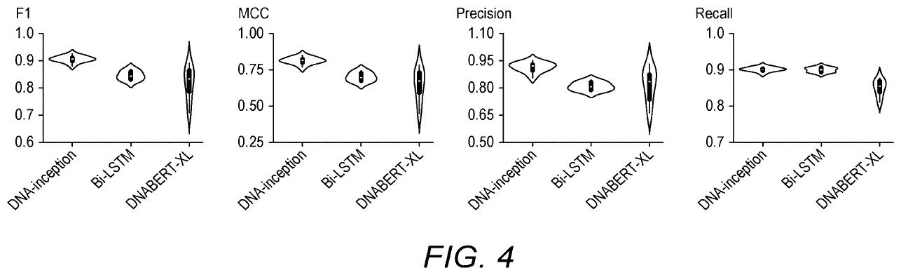

In this disclosure, there is provided a deep neural network model, referred to here as DNA-Inception. DNA-Inception is merely a label used to refer to the specific neural network configuration proposed according to an example implementation of the present disclosure. Although the system is not restricted to using DNA-Inception to perform classification or regression tasks, any other derivable parametric function is contemplated for use. It has however been shown that the specific configuration of DNA-Inception in initial benchmark analysis achieves good performance in comparison to two other state-of-the-art approaches. More details on the preferred neural network configuration and alternative machine learning models contemplated will be provided below in the context of FIGS. 3 and 4.

Once the model has been trained, as explained above in the context of FIG. 1, the next step is to interpret the trained model to discover the cancer-enriched motifs, described here as the model explanation method and illustrated as steps 203 to 207.

To interpret the predictions of the selected model, a saliency approach is proposed, such as Integrated Gradient, to assign a feature attribution score, aᵢ∈A, to each nucleotide in an input sequence of cancer specific alternative splicing events, i.e., aᵢ→xᵢ∈x where i∈{1, 2, . . . , L}, x∈X, L=len(x) and y=cancerous. Nucleotides that contribute positively to cancerous class are assigned positive values and vice versa.

In this example implementation, Integrated Gradient is used, however other model explanation methods that explain the relationship between inputs and outputs based on what the model learned can be leveraged such as Guided Backprop, Occlusion Maps, etc.

These and other suitable interpretation methods will be understood by the skilled person, however, what is important is that that the interpretation method is able to identify features of the input which result in the prediction from the trained network model. In other words, which part of the DNA sequence links to a cancerous alterative splice event, that is, which features are learnt to accurately predict the class from the DNA sequence.

Once the model has been interpreted to identify how the input positively contributes to the classification, the workflow uses that interpretation to select high-attribution regions, step 204.

For each input sequence x∈X where y=cancerous, the system selects DNA regions of length≥M, where all nucleotide within the region has attribution scores greater than a threshold value t. For example the threshold t can be defined based on the minimum positive attribution score or the average attribution score of the input sequence.

Other mechanisms of grouping the features are contemplated, for example the scores may be compared to the scores for neighbouring features, compared to metrics for the whole sequence, compared to metrics for the region or metrics calculated for the region.

Similarly, we describe here that the features utilised may be each individual nucleotide for the sequence, however, it will be understood that the model may also be trained using additional features such as groups of nucleotides or neighbouring nucleotide information. What is important is that the model learns to classify cancerous or healthy related DNA sequences based on features of those sequences with those features being interpretable to lead to motif discovery.

There follows a series of optional filtering steps to ensure the list of high attribution regions are statistically relevant and are likely to be associated with an alternative splicing event corresponding to a cancerous tumour. It will be understood that these inference and correction steps may be performed in different orders or may be replaced or supplemented with further steps which will most likely be statistical in nature.

Preferably hypergeometric test is applied, step 205, for statistical inference and to apply testing corrections.

In the hypergeometric test the number of positive (i.e., cancerous) sequences containing cancer-enriched motifs is assumed to follow a Hypergeometric distribution X~Hypergeometric(N, K, n). The probability mass function (pmf) of a random variable X following the hypergeometric distribution is,

$$P ( X = k ) = \frac{( \begin{matrix}K \\ k\end{matrix} ) ( \begin{matrix}N - K \\ n - k\end{matrix} )}{( \begin{matrix}N \\ n\end{matrix} )}$$

where N is the total number of sequences (i.e., population size), K is the total number of cancerous sequences, n is the number of sequences with a specific motif, and k is the number of cancerous sequences containing that specific motif.

A hypergeometric test is performed to obtain significantly enriched motifs in cancer and filter out the ones that are common in both healthy and cancerous alternative splicing event sequences.

For each motif, the hypergeometric test first computes the probability of drawing without replacement P(X≥k) sequences containing that motif under the null hypothesis, i.e., such a motif is equally likely to appear in healthy as well as in cancerous AS events. To this end, the survival function sf is leveraged, i.e., the inverse of the cumulative distribution function cdf, i.e., sf=1−cdf=1−P(X<k). If the p-value (i.e., output probability) is sufficiently low after adjusting for multiple testing, then it is concluded that the null hypothesis is unlikely and it is rejected. That motif is considered significantly enriched in cancer events, and it is added it to a motif output list.

Various methods can be used for multiple testing corrections, e.g., Bonferroni correction, Benjamini and Hochberg false discovery rate, etc.

Once we have our preliminary motif output list, corresponding to the high attribution regions, the workflow optionally merges similar motifs, step 206. Merging similar motifs may be performed using, for example, the implementation of PairwiseAligner( ) class from the biopython library. That is, the motifs may be merged using a suitable pairwise sequence aligning technique. Sequence alignment is the process of arranging two or more sequences (of DNA, RNA or protein sequences) in a specific order to identify the region of similarity between them. As would be well understood by the skilled person, pairwise sequence alignment compares two sequences at a time and provides best possible sequence alignments. Pairwise is easy to understand and exceptional to infer from the resulting sequence alignment.

For each query motif, if it can be aligned to an existing one, the maximum score alignment is chosen where internal gaps are prohibited to maintain the integrity of motifs. If the approach fails to align a motif, then it is added it to the output list. Other alignment techniques or implementations can be leveraged to perform the task.

The merged list of motifs is then filtered to remove less frequent motifs, step 207. The system filters motifs which occur in cancer sequences less than a user predefined number, i.e., min_occurrence. In this way, we can be more confident that each motif is statistically significant and representative of a DNA sequence corresponding to a cancerous alternative splicing event.

The output of the system is a list of cancer-enriched motifs, step 208. In further example implementations, the output may also comprise the genomic coordination of the motifs, and the list of genes and transcripts where those motifs occur.

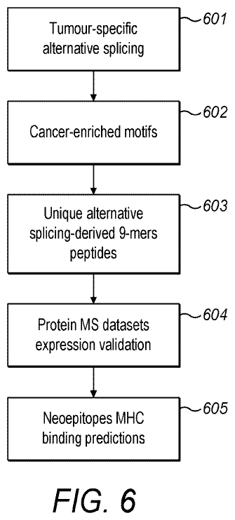

This final output motif list can be used for cancer diagnostics. In detail, when obtaining a new RNA-seq sample, all alternative splicing event sequences are enlisted, and the trained model can be run, e.g., to predict whether each event belongs to cancer or healthy tissue. If nucleotide sequences that have higher likelihood of occurring in cancer samples are found, the sample is diagnosed as cancerous. Further, the motif discovery algorithm may be run to enlist all significant cancer-enriched motifs, and compare the new motifs with the previously found motifs to obtain all previously verified alternative splicing-derived neoepitopes with verified immunogenicity. If nucleotide sequences that have higher likelihood of occurring in cancer are not found, a list of alternative splicing-derived neoepitope or neoantigen candidates can be obtained by extracting unique k-mer peptides (where k≥9) covering the cancer-enriched motifs and filtering out peptides that are present in a non-cancer proteomic dataset. The potential neoepitopes or neoantigens may then be confirmed using protein mass spectrometry (MS) databases obtained from various tumour types (such as Clinical Proteomic Tumor Analysis Consortium (CPTAC) MS data). The immunogenicity of the resultant neoepitopes or neoantigens can be predicted using existing tools to extract neoepitope or neoantigen candidates that can be leveraged in cancer immunotherapy. An example workflow is illustrated in FIG. 6.

As well as for use in cancer diagnostics, the motifs may be used to identify neoantigens based on the cancer-enriched motifs.

Splicing variations in cancer and the resulting motifs are a rich source of neoepitope or neoantigen candidates. AS based neoantigens can be leveraged in the development of novel immunotherapies such as personalised cancer vaccines. To leverage cancer-enriched motif for therapeutic purposes, further validation experiments may be used to check the immunogenicity of the resulting peptides covering those motifs. The goal is to elicit a T-cell response in patient samples when pulsed with peptides covering the motifs.

Obtaining a list of alternative splicing-derived neoepitope or neoantigen candidates involves the steps of: extracting k-mer peptides (where k≥9) covering each of the cancer-enriched motif, filtering out the peptides that are present in non-cancer proteomic dataset; confirming potential neoepitopes or neoantigens in protein mass spectrometry (MS) databases obtained from various tumour types (such as Clinical Proteomic Tumor Analysis Consortium (CPTAC) MS data); and running an existing tool to predict whether the neoepitopes or neoantigens bind to major histocompatibility complex (MHC) to check their immunogenicity. Neoepitopes or neoantigens which provoke an immune response can be leveraged in cancer immunotherapies. These verification steps are well-described in literature (for example, Kahles et al. “Comprehensive analysis of alternative splicing across tumors from 8,705 patients” Cancer cell 34.2 (2018): 211-224). Furthermore, peptides derived from newly identified alternative splicing events can be leveraged in identifying novel neoepitope or neoantigen candidates in MS data from eluted peptide-MHC complexes that have not been previously detected and are thus not present in standard MS reference databases. Finally, the candidate neoepitopes (or neoantigens) can be leveraged in cancer immunotherapy vaccinations to help immune system recognise such antigens or epitopes and eliminate cancer cells which produce them.

Samples for immunopeptidomes and Peripheral Blood Mononuclear Cells (PBMCs) are available and may be used to complete this step. Such validation experiments are well known in the art.

Moreover, the algorithms and workflow can be adapted to identify significant motifs from novel splicing variations in inherited diseases, such as autism spectrum disorder, which may facilitate the development of potential treatments.

FIG. 3 illustrates a model specifically developed to address the specific classification task needed in the proposed workflow. As indicated above, here we refer to the specifically configured trained model as DNA-Inception.

DNA-Inception is an example of a derivable parametric model which can be leveraged in the workflow. The model was developed to attest the validity of the proposed system.

The DNA-Inception model is a multi-kernel-size convolutional network for one-dimensional (1D) signals which was inspired by Inception v1. Inception was developed to solve pattern recognition problems in computer vision and is set out in Szegedy, Christian, et al. “Going deeper with convolutions.” Proceedings of the IEEE conference on computer vision and pattern recognition. 2015. Inception allowed for interpretability while giving high performance.

DNA-Inception, i.e., the example implementation proposed herein, comprises an embedding layer which is represented by a matrix W∈ [symbol] ^{n×d}, where n is the vocabulary size and d is a customized embedding dimension. Considering an input sequence x of length L nucleotides 301, the sequence is first encoded using a tokeniser encoding scheme. Tokenisation replaces elements in the sequence with tokens to facilitate analysis. As will be understood, any suitable encoding technique could be used to facilitate vector or matrix computation.

Then, the embedding layer 302 operates matrix multiplication xW to map each nucleotide into [symbol] ^{d} space, the shape of the output matrix is xd. The embedding layer is followed by n 1D convolutional blocks 303, where n∈{1, . . . , N}. Each convolutional block consists of (i) multiple 1D convolution layers 304 with kernel size equals to 1; these layers are added to improve computational efficiency and reduce the number of parameters, followed by (ii) another set of 1D convolutions 305 with variable kernel sizes, to capture dynamics at different scales in input signals, then we have (iii) 1D max pooling layers 306. All output tensors from the final convolution block are concatenated 307 and passed through a series of fully connected layers 308.

The proposed deep learning-based approach is used for classifying RNA splicing in cancerous and normal tissues as opposed to existing approaches which rely on enumerating all novel splicing in tumours by one-to-one comparisons to all splicing events that occur in normal samples. Using a deep learning-based model helps improve precision in identifying common cancer-specific splicing patterns in pan-cancer analysis.

As mentioned, multiple suitable machine learning techniques could be used provided it is possible to interpret the trained model to identify the positively contributing features (i.e., the contribution of each nucleotide to the classification).

To demonstrate this, multiple comparisons were performed between the configured machine learning model specifically disclosed herein, i.e., DNA-Inception, the pattern recognition CNN described above, and other models.

First, DNA-Inception is compared, the model we developed in this invention, to the state-of-the-art DNABERT and a baseline model we developed using Bi-LSTM. DNABERT is published in Ji, Yanrong, et al. “DNABERT: pre-trained Bidirectional Encoder Representations from Transformers model for DNA-language in genome.” Bioinformatics 37.15 (2021): 2112-2120. Second, the proposed system is compared to existing methods and pipelines for identifying alternative splicing-based cancer neoantigens. Third, the system is compared to existing methods for identifying cancer motifs.

DNABERT is a pre-trained Bidirectional Encoder Representations from Transformers model for DNA-language in genome. DNABERT is a general-purpose model which can be trained and fine-tuned on any classification task given that the inputs are DNA sequences. The authors provided a pretrained model where the whole human genome was leveraged in the pretraining step. The authors stated that pretraining DNABERT took approximately 25 days on 8 NVIDIA 2080Ti GPUs. In an initial benchmark analysis using ExonSkipDB dataset, we trained and evaluated DNABERT-XL which was developed to handle DNA sequences longer that 512 nucleotides. DNABERT uses k-mer sequences as inputs. The authors reported the best performance was achieved when k=6, hence this value was used for k in the benchmark analysis.

Table 1, below, and FIG. 4 show a performance evaluation over 10 runs, in each run the dataset is randomly split into 80% for training and validation and 20% for testing. FIG. 4 shows violin plots of the performance of DNABERT-XL, Bi-LSTM and DNA-Inception over 10 runs. The 3 methods were benchmarked for classifying cancerous and healthy AS sequences from the ExonSkipDB dataset.

DNABERT-XL consistently gives lower average performance values and higher variances in comparison to DNA-Inception. Additionally, DNABERT-XL requires 24 GB for GPU RAM while training. DNA-Inception outperforms DNABERT-XL in performance, model complexity in terms of total number of trainable parameters, training time and GPU requirement.

Bi-LSTM is a baseline model which consists of an embedding layer and bi-directional long Short-term memory network followed by a fully connected last layer. Bi-LSTM is a common approach for handling genomic sequences. For this baseline approach k-mer sequences were used as inputs where k=6 was set. During initial benchmarking, significantly improved performance was noticed when using k-mer sequences instead of a character-level approach. Empirically, setting k=6 gave the best results in comparisons to k=3, 4 or 5. Again, the disclosed DNA-Inception outperforms Bi-LSTM in initial benchmark analysis.

Lastly, when benchmarking the three models, the same evaluation criteria and stopping metric was set.

The following table 1 shows evaluating the three models based on their training time, complexity in terms of total trainable parameters and GPU model. The average and standard deviation of training times over 10 different runs are shown.

| | DNA-Inception | Bi-LSTM | DNABERT-XL |
|---|---|---|---|
| Average training | 230.796 ± | 15288.692 ± | 16276.255 ± |
| time in seconds | 42.781 | 2373.51 | 2765.780 |
| Total number of | 199,874 | 287,170 | 90,962,690 |
| trainable parameters | | | |
| GPU | 4 × Geforce | 4 × Geforce | 4 × TITAN RTX |
| | RTX 2080 Ti | RTX 2080 Ti | (24 GB RAM) |
| | (11 GB RAM) | (11 GB RAM) | |

According to the example implementation proposed, a deep learning system is described for identifying cancer-enriched sequence motifs from novel splicing variations in tumours. To this end, a neural network model was developed based on one-dimensional (1D) convolution. The model classifies cancerous and normal AS events. Then, the algorithm highlights important input nucleotides in cancerous events based on model predictions using Integrated Gradient saliency approach. After that, our algorithm applies hypergeometric test and additional merging and filtering techniques to identify a set of DNA regions of significantly represented motifs in cancer specific AS events. The set of cancer-enriched motifs can be leveraged in further downstream analysis to obtain AS-based neoantigen candidate.

Furthermore, the approach generalises to new unseen AS events and does not require repetitive manual comparisons between tumour and healthy samples, as the model is trained once on large number of samples and is able to distinguish normal and cancer specific AS events with an F1 and recall scores greater than 90%.

The proposed system identifies cancer-enriched motifs from splicing variations in tumour and is not limited to the so-called mutation signatures. Mutations in tumour can disrupt AS and result in novel splicing variations. Thus, as an input to the system, DNA mutations are considered which are present in tumour samples to gain a comprehensive understanding of splicing variations and the accompanied cancer-enriched motifs. Thus, the system provides more comprehensive view for eliciting cancer-enriched motifs. Motifs based on tumour splicing variations have not been examined before.

Datasets which offer AS annotations for tumour and healthy samples are collected based on various sequencing technologies, e.g., whole-genome and whole-exome sequencing. The system may be more powerful when applied to AS datasets that was collected based on reads overlapping the coding and non-coding regions of the DNA, i.e., whole-genome sequencing. The system may be less powerful when applied to AS datasets that are based on coding exomes alone, i.e., whole-exome sequencing, since we can potentially miss cancer-enriched motifs from the non-coding regions (i.e., introns). Nevertheless, the system still demonstrates utility in this regard.

Various datasets are generated using varying sequencing depths which may introduce undesired biases in AS read evidence. This can be avoided by ensuring that the initial datasets meet certain quality criteria. Otherwise, variance in the results is expected due to different sequencing run, depth, and quality.

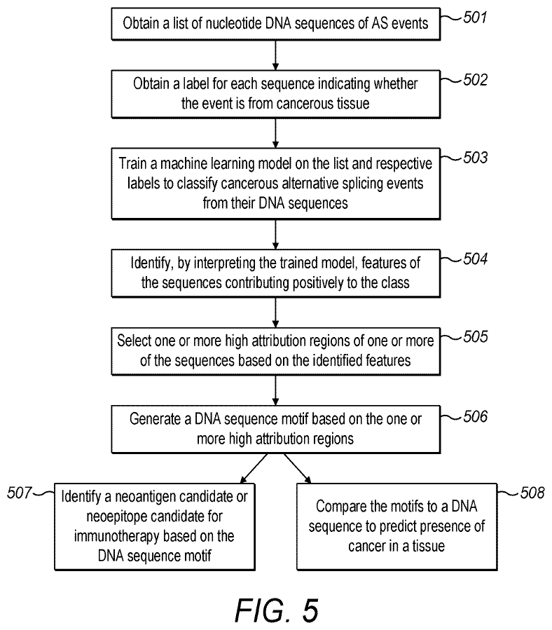

For completeness, the present disclosure provides a computer-implemented method for identifying DNA sequence motifs indicative of a disease from alternative splicing events. FIG. 5 illustrates the steps of an example implementation in the form of a flow diagram. The method comprises, at step 501, obtaining a list of nucleotide DNA sequences, each sequence representing a sequence of an alternative splicing event. At step 502, the method obtains a label for each sequence, the label indicating if the sequence is associated with a healthy sample or a diseased sample. Next, at step 503, the implementation trains a machine learning model on the list and respective labels to classify alternative splicing events from their DNA sequences based on whether they occur in healthy or diseased samples. From the trained model, the implementation then is capable of producing cancer-enriched motifs for use in neoantigens for immunotherapy and diagnostics. At step 504, the process identifies, by interpreting the trained model, features of the sequences contributing positively to indicate whether the sequences occur in healthy or diseased sample. Then, at step 505, selects one or more high attribution regions of one or more of the sequences based on the identified features, wherein the high attribution region indicates a region of the sequence having positively contributing features. At step 506, the process generates a DNA sequence motif based on the one or more high attribution regions. Optionally, at step 507, the motif is used to identify a neoantigen candidate or neoepitope candidate for immunotherapy based on the DNA sequence motif, or at step 508 the motif is compared to a DNA sequence to predict presence of cancer in a tissue based on alternative splicing events.

It has been described above how the DNA sequence motif may be utilised in the formation of a neoantigen for vaccine development, or as a diagnostic.

Various bioinformatics approaches may be used to translate the DNA sequence of the motif into the corresponding peptide sequence to predict the neoantigen or neoepitope. Machine learning-based software solutions are available for the in-silico prediction and identification of optimal immunogenic neoantigens or neoepitopes for personalised cancer immunotherapy. For example, neoantigen prediction systems (such as NeoAntigen Quest (NAQ) and NEC Immune Profiler (NIP)) combine transcriptomic and proteomic data to identify immunogenic neoantigen candidates or neoepitope candidates from DNA sequence information with high accuracy predictive power as to whether a neoantigen or neoepitope will be naturally processed and presented on the surface of a tumour cell, and thus, the immunogenic potential (or “immunogenicity”) of said neoantigen candidate or neoepitope candidate.

Obtaining a list of alternative splicing-derived neoepitope or neoantigen candidates involves the steps of: extracting k-mer peptides (where k≥9) covering each of the cancer-enriched motif, filtering out the peptides that are present in non-cancer proteomic dataset; confirming potential neoepitopes or neoantigens in protein mass spectrometry (MS) databases obtained from various tumour types (such as Clinical Proteomic Tumor Analysis Consortium (CPTAC) MS data); and running an existing tool to predict whether the neoepitopes or neoantigens bind to major histocompatibility complex (MHC) to check their immunogenicity. Neoepitopes or neoantigens which provoke an immune response can be leveraged in cancer immunotherapies. These verification steps are well-described in literature (for example, Kahles et al. “Comprehensive analysis of alternative splicing across tumors from 8,705 patients” Cancer cell 34.2 (2018): 211-224). Furthermore, peptides derived from newly identified alternative splicing events can be leveraged in identifying novel neoepitope or neoantigen candidates in MS data from eluted peptide-MHC complexes that have not been previously detected and are thus not present in standard MS reference databases. Finally, the candidate neoepitopes (or neoantigens) can be leveraged in cancer immunotherapy vaccinations to help immune system recognise such antigens or epitopes and eliminate cancer cells which produce them.

FIG. 6 illustrates the steps of how a list of alternative splicing-derived neoepitope or neoantigen candidates is obtained, in the form of a flow diagram comprising steps 601 to 605. The flow diagram illustrates an alternative splicing-derived neoantigens workflow. The process begins with the tumour-specific alternative-splicing (step 601). At step 602, cancer-enriched motifs produced as a result of tumour-specific alternative splicing events are identified using the method for identifying DNA sequence motifs as described herein. At step 603, k-mer peptides (where k≥9) covering the cancer-enriched motifs are extracted. Peptides that are present in a non-cancer proteomic dataset are filtered out to obtain the unique alternative splicing-derived k-mer peptides. At step 604, the potential neoepitopes or neoantigens are confirmed using protein mass spectrometry (MS) databases obtained from various tumour types (such as Clinical Proteomic Tumor Analysis Consortium (CPTAC) MS data). Finally, step 605 involves the prediction of the immunogenicity of the resultant neoepitopes or neoantigens. Neoepitopes or neoantigens which bind MHC are predicted to provoke an immune response and can therefore be leveraged in cancer immunotherapies.

The neoantigen candidates or neoepitope candidates that are identified by such quantitative statistical analysis may represent viable vaccine targets that may instigate a broad T-cell immune response, and may be used in vaccine design and creation. Once the sequence of the neoantigen or neoepitope is obtained (for example, via mass spectrometry), the peptides can be synthesised, i.e., via in vitro solid- or liquid-phase peptide synthesis methods, and purified, i.e., via classical separation-based methods. These methods will be well known in the art.

The term “neoepitope” as used herein refers to any part of a neoantigen that is recognised by any antibodies, B cells, or T cells. A “neoantigen” refers to a molecule capable of being bound by an antibody, B cell or T cell, and may be comprised of one or more neoepitopes. As such, the terms neoepitope and neoantigen may be used interchangeably herein.

Such an approach will be validated against clinically relevant neoantigens or neoepitopes to check the immunogenicity of the resulting peptides, and ensure that the chosen neoantigen or neoepitope is successful in eliciting a T-cell response in patient samples. This can be carried out using immunogenicity assays (for example, via quantitative enzyme-linked immunosorbent assays (ELISA)) in which patient samples are pulsed with peptides covering the motifs.

Alternatively, further immunopeptidome analysis can be used to identify which peptides from alternative splicing events are at least presented on the tumour cell surface. Neoantigens or neoepitopes are presented on the tumour cell surface by class-I and class-II major histocompatibility complex (MHC) molecules. The peptides associated with and presented by MHC molecules are collectively referred to as the immunopeptidome. The immunopeptidome can be extracted from cell or tissue samples using various isolation techniques that will be known in the art, and immunoaffinity purification of the MHC molecules followed by release of the peptides from the isolated MHC molecules. The resultant purified peptides can then be analysed via mass spectrometry. The mass spectrometry derived peptides that cannot be mapped to the healthy immunopeptidome and can be mapped to novel splicing variations in cancer.

The term vaccine relates to a biological preparation that provides active acquired immunity to a particular disease, in this case a cancer or tumour. Typically, the vaccine contains an agent, or “foreign” agent, that resembles a neoantigen or neoepitope from the cancer or tumour cell surface. Such a foreign agent would be recognised by a vaccine-receiver's immune system, which in turn would destroy said agent and develop “memory” against the cancer or tumour, inducing a level of lasting protection against future disease caused by reoccurrence of the same cancer or tumour. Through the route of vaccination, including through a vaccine created by a method of the present invention, it is envisaged that once the vaccinated subject again encounters the same cancer or tumour of which said subject was vaccinated against, the individual's immune system may thereby recognise said cancer or tumour and elicit a more effective immune defence.

The active acquired immunity that is induced may be humoral and/or cellular. Humoral immunity refers to a response involving B cells which produce antibodies that specifically bind to neoantigens or neoepitopes, or any future neoantigens or neoepitopes, corresponding to those within the administered vaccine. B cells, each expressing a unique B cell receptor (BCR), recognise neoantigens or neoepitopes in their native form. Upon this recognition and further interaction with other cells of the immune system, the activated B cell can differentiate into a plasma cell specialised to secrete antibodies against the encountered neoantigen or neoepitope. The term antibody refers to an immunoglobulin (Ig) that is used by the immune system to specifically identify and neutralise foreign antigens. A subset of these B-cell derived plasma cells become long-lived antigen-specific memory B cells, as would be well understood by the skilled person.

Cellular immunity, meanwhile, can be broken into two distinct arms. The first involves helper T cells, or CD4+ T cells, which produce cytokines and orchestrate the activity of other immune cells in the immune response. The second involves killer T cells, also known as cytotoxic T lymphocytes (CTLs), or CD8+ T cells, which are cells capable of recognising neoantigens or neoepitopes presented on the surface of cancer or tumour cells and eradicate the cancer or tumour cells. In contrast to B cells, T cells only recognise neoantigens or neoepitopes that have been processed into peptides and have been loaded onto the MHC molecule and presented at the cell surface. CD4+ T cells interact with MHC class-II molecules, and are responsible for orchestrating the immune response, recognizing foreign antigens (i.e., neoantigens or neoepitopes), activating various parts of the immune system and activating B cells and CD8+ T cells. CD8+ T cells interact with MHC class-I receptors on the surface of antigen-presenting cells (APCs) and target cells, which display antigenic peptide fragments produced by proteasomal degradation. As would be understood by the skilled person, following an immune response, a subset of both CD8+ T cells and CD4+ T cells may remain as memory T cells, contributing to the acquired adaptive immunity, and allowing for a faster and stronger response to any future recognition of the same neoantigens or neoepitopes.

It is envisaged that a vaccine created by a method of the present invention may be an epitope-based (i.e., neoepitope-based) vaccine, or in other words, is comprised of one or more epitopes. Epitope-based vaccines (EVs) make use of short antigen-derived peptides corresponding to immune epitopes, which are administered to trigger a protective humoral and/or cellular immune response. EVs potentially allow for precise control over the immune response activation by focusing on the most relevant (i.e., immunogenic and conserved) antigen regions. Experimental screening of large sets of peptides is time-consuming and costly; therefore, in-silico methods that facilitate T-cell epitope mapping of protein antigens are paramount for EV development. The prediction of T-cell epitopes focuses on the presentation of peptides on the cancer or tumour cell surface by proteins encoded by the MHC.

The neoantigens or neoepitopes of the present invention may interact with MHC class-I and/or MHC class-II molecules to induce a CD8+ T cell and/or CD4+ T cell response, respectively. There may be at least one neoantigen or neoepitope that interacts with MHC class-I, and at least one neoantigen or neoepitope that interacts with MHC class-II.

Any or all steps of the methodology may be implemented in a local, remote or cloud computing device. The trained model and its associated parameters may be stored centrally, i.e., on the cloud. Methods and processes described herein can be embodied as code (e.g., software code) and/or data. The models, methodologies and algorithms may be implemented in hardware or software as is well-known in the art of machine learning. For example, hardware acceleration using a specifically programmed Graphical Processing Unit (GPU) or a specifically designed Field Programmable Gate Array (FPGA) may provide certain efficiencies. For completeness, such code and data can be stored on one or more computer-readable media, which may include any device or medium that can store code and/or data for use by a computer system. When a computer system reads and executes the code and/or data stored on a computer-readable medium, the computer system performs the methods and processes embodied as data structures and code stored within the computer-readable storage medium. In certain embodiments, one or more of the steps of the methods and processes described herein can be performed by a processor (e.g., a processor of a computer system or data storage system).

Generally, any of the functionality described in this text or illustrated in the figures can be implemented using software, firmware (e.g., fixed logic circuitry), programmable or nonprogrammable hardware, or a combination of these implementations. The terms “component” or “function” as used herein generally represents software, firmware, hardware, or a combination of these. For instance, in the case of a software implementation, the terms “component” or “function” may refer to program code that performs specified tasks when executed on a processing device or devices. The illustrated separation of components and functions into distinct units may reflect any actual or conceptual physical grouping and allocation of such software and/or hardware and tasks.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1: A computer-implemented method of training a machine learning model for use in identifying sequence motifs indicative of a disease from alternative splicing events, the method comprising:
obtaining a list of nucleotide sequences, each sequence of the list of nucleotide sequences representing a sequence of an alternative splicing event;
obtaining a label for each sequence, the label indicating whether the each sequence is associated with a healthy sample or a diseased sample; and,
training a machine learning model on the list of nucleotide sequences and respective labels for each sequence to classify alternative splicing events from corresponding sequences based on whether they occur in healthy or diseased samples.

2: The method according to claim 1, wherein each respective label indicates whether the each sequence is associated with a sample of healthy tissue or cancerous tissue, and wherein the machine learning model is trained to classify alternative splicing events from corresponding sequences based on whether they occur in healthy or cancer tissues.

3: The method according to claim 1, wherein each respective label indicates whether the each sequence is associated with an inherited disease, and wherein the machine learning model is trained to classify alternative splicing events from corresponding sequences based whether the corresponding sequences relate to an inherited disease.

4: The method according to claim 1, wherein the machine learning model is a neural network.

5: The method according to claim 1, the method further comprising:
identifying, by interpreting the trained machine learning model, features of sequences of the list of nucleotide sequences contributing positively to indicate whether the sequences occur in healthy or diseased samples;
selecting one or more high attribution regions of one or more of the sequences based on the identified features, wherein the high attribution region indicates a region of the sequence having positively contributing features; and
outputting a sequence motif based on the one or more high attribution regions.

6: The method according to claim 5, wherein each feature is a nucleotide of the sequence, wherein the sequence is a DNA sequence.

7: The method according to claim 6, wherein the selecting comprises:
generating a feature attribution score for each nucleotide in a respective sequence based on a contribution of that nucleotide to the classification; and,
selecting a region of the sequence based on the score for each nucleotide.

8: The method according to claim 7, wherein the selecting a region of the sequence based on the score for each nucleotide comprises:
comparing the score for each nucleotide in a candidate region to a threshold;
calculating an average of scores for the nucleotides in a candidate region;
calculating an average of scores for the sequence and comparing the score for each nucleotide in a candidate region to the average of scores for the DNA sequence.

9: The method according to claim 5, wherein the selecting comprises:
applying a hypergeometric test to the one or more high attribution regions to obtain significant regions present in the sequences associated with a diseased sample label and filter out regions common to the sequences associated with a diseased sample label and the sequences associated with a healthy sample label.

10: The method according to claim 5, further comprising:
merging a plurality of the selected one or more high attribution regions using pairwise sequence alignment.

11: The method according to claim 5, further comprising:
filtering the selected one or more high attribution regions based on an amount of occurrences of each region in DNA sequences associated with a diseased sample label.

12: The method according to claim 5, further comprising:
identifying a neoantigen candidate or neoepitope candidate for immunotherapy based on the sequence motif.

13: A computer readable medium having computer executable instructions stored thereon for implementing the method of claim 1.

14: A method of creating a vaccine, comprising:
selecting one or more predicted immunogenic candidate amino acid sequences covering a sequence motif output according to the method of claim 5 for inclusion in a vaccine; and
synthesising one or more amino acid sequences or encoding the one or more amino acid sequences into a corresponding DNA or RNA sequence and/or incorporating the DNA or RNA sequence into a genome of a bacterial or viral delivery system to create a vaccine.

15: A method of predicting likelihood of a tissue sample being cancerous, comprising:
retrieving a sequence of a tissue sample;
analysing the sequence of the tissue sample for similarity to the sequence motif output according to the method of claim 5; and,
predicting likelihood of a tissue sample being cancerous based on the analysis.

16: The method according to claim 4, wherein, the neural network is a multi-kernel-size convolutional neural network for 1-dimensional signals.
